(19) United States
(12) Patent Application Publication
(10) Pub. No.: US 2023/0033522 A1
(43) Pub. Date: Feb. 2, 2023

(54) ANASTOMOTIC LEAKAGE SENSOR AND ANALYSIS OF PREDICTIVE PARAMETERS FOR DETECTING AN ANASTOMOTIC LEAKAGE

(71) Applicant: Covidien LP, Mansfield, MA (US)

(72) Inventors: Osvaldo A. Barrera, Madison, CT (US); Jeffrey A. Miller, East Haven, CT (US); Banu Akar, Hamden, CT (US); Daniel C. Broom, Branford, CT (US)

(21) Appl. No.: 17/391,343

(22) Filed: Aug. 2, 2021

(51) Int. Cl. A61B 17/11; G16H 40/67

(52) U.S. Cl. CPC A61B 17/11; G16H 40/67; A61B 2017/00035

(57) ABSTRACT

A system for detecting an anastomotic leak includes a sensor assembly implanted at an anastomosis site. The system also includes a reader configured to receive sensor signals from the sensor assembly and a computing device configured to communicate with the reader. The computing device may be configured to analyze the sensor signals to determine a status of the anastomosis site.

### BACKGROUND

Colectomy procedures to remove a portion of a large intestine are performed to treat various conditions, such as cancer, diverticulitis, trauma, and inflammatory bowel disease. Intestinal anastomosis may be performed using various techniques and medical devices, such as end-to-end stapling devices, which are used in colorectal surgery to connect portions of colon, large intestines, etc. Anastomosis may result in post-operative complications, such as anastomotic leaks. An anastomotic leak is one of the most serious surgical complications that can develop following colorectal surgery. The anastomotic leak rate varies from about 4% to about 9%, depending on the colorectal procedure, with the highest percentages associated with low anterior resections.

The root causes of the leaks are not completely known, but several factors increase the risk of leaks, such as certain medical conditions, including, diabetes, ischemia, infection, etc. Leaks are a major post-operative complication and lead to peritonitis, sepsis, and morbidity. Patients who develop chronic leaks need endoscopic drainage, surgical intervention, and clinical monitoring. Monitoring is critical to determine the status of the leak. Depending on the extent of the leak, different management methods may be used, such as drainage, extensive laparotomy, as well as an open surgery, which has an elevated risk of permanent ostomy, especially for low rectal resection patients.

The current management of complications is reactive, treating a complication long after it occurred and advanced into systemic complication, such as sepsis. Such approaches have severe outcomes for both patient and hospital, ranging from reduced quality of life for the patient to increased resource use in treating the patient. Anastomotic leaks present a major clinical problem and can increase the medical costs by approximately $17,000 and almost double the length of the hospital stays (from about 8.4 days for patients with no leaks to about 14.9 days). Leak patients also have higher post-operative infection rates and spend on average 7 more days in the hospital, compared with non-leak patients. The extra days result in additional costs to the hospital. Thus, there is a need for a more proactive approach to anastomotic leak management through earlier diagnosis and intervention.

### SUMMARY

The present disclosure provides a system and method for continuously monitoring of stability of the anastomosis and detection of anastomotic leaks at their earliest onset, thereby giving the physician a greater chance to minimize or eliminate the anastomotic leaks. This would contribute to reducing patient suffering, costs, and length of hospital stay.

According to one embodiment of the present disclosure, an implantable sensor, which may be a pH sensor, is implanted at an anastomosis site. The sensor may be attached to the anastomosis site using a biodegradable securing device, which may be tissue adhesive, one or more fastener, sutures, or any other suitable device or composition, and combinations thereof. After the biodegradable securing device degrades or dissolves, which releases the sensor from the site, the sensor is removed by a clinician for potential analysis and disposal. The sensor outputs sensor data which is analyzed by a computing device to determine the status of the anastomosis site.

According to one embodiment of the present disclosure, a system for detecting an anastomotic leak is disclosed. The system may include a sensor assembly implanted at or in proximity to (e.g., from 1 cm to about 10 cm away from) an anastomosis site. The system also includes a reader configured to receive sensor signals from the sensor assembly and a computing device configured to communicate with the reader. The computing device may be configured to analyze the sensor signals to determine a status of the anastomosis site.

Implementations of the above embodiment may include one or more of the following features. According to one aspect of the above embodiment, the sensor assembly may include: a sensor element configured to measure pH of the anastomosis site. The sensor assembly further may include a power module coupled to the sensor element via a cable. The power module may be implanted subcutaneously and outside the peritoneum. The computing device may be configured to compare the sensor signals to a pH range. The computing device may be configured to detect a pattern in the sensor signals indicative of an anastomotic leak. The sensor assembly may include a substrate having a first surface and a second surface and a substantially planar shape. The substrate may include at least one opening configured to be used as an attachment point. The computing device may be configured to communicate with a remote server. The remote server may be configured to analyze the sensor signals to determine the status of the anastomosis site. The reader is further configured to wirelessly transmit power to the sensor assembly.

According to one embodiment of the present disclosure, a method for detecting an anastomotic leak is disclosed. The method may also include implanting a sensor assembly at an anastomosis site and wirelessly receiving sensor signals from the sensor assembly at a reader. The method may also include analyzing the sensor signals to determine a status of the anastomosis site.

Implementations of the above embodiment may include one or more of the following features. According to one aspect of the above embodiment, the method further may include: measuring pH at the anastomosis site. The method may also include implanting a sensor element at the anastomosis site. The method may further include implanting a power module subcutaneously and outside the peritoneum, where the power module is coupled to sensor element via a cable. The method may also include comparing the sensor signals to a pH range. The method may also include detecting a pattern in the sensor signals indicative of an anastomotic leak.

According to one embodiment of the present disclosure, a sensor assembly for detecting an anastomosis leak is disclosed. The sensor assembly may include a substrate having a first surface and a second surface and a substantially planar shape; and a sensor element configured to measure pH disposed on the substrate.

Implementations of the above embodiment may include one or more of the following features. According to one aspect of the above embodiment, the substrate may include at least one opening configured to be use as an attachment point. The sensor assembly may also include an adhesive disposed on at least one of the first surface or the second surface.

### BRIEF DESCRIPTION OF THE DRAWINGS

Embodiments of the present disclosure are described herein with reference to the accompanying drawings, wherein:

FIG. 1 is a schematic view of an anastomotic leak detection system according to an embodiment of the present disclosure;

FIG. 2 is a schematic view of a power module of a sensor assembly according to an embodiment of the present disclosure;

FIG. 3 is a schematic view of a system for monitoring the anastomotic leak detection system according to an embodiment of the present disclosure;

FIG. 4 is schematic view of an anastomotic leak sensor according to an embodiment of the present disclosure; and

FIG. 5 is a schematic illustration of a method for retrieving the anastomotic leak sensor of FIG. 3.

### DETAILED DESCRIPTION

Embodiments of the presently disclosed system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IOT device, or a server system.

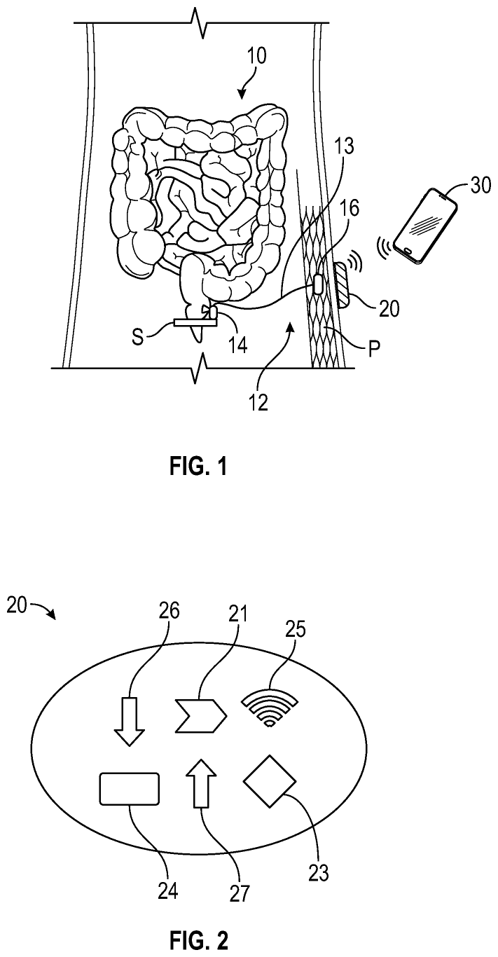

With reference to FIG. 1, a system 10 for detecting an anastomotic leak is shown. The system 10 includes a sensor assembly 12 implanted at an anastomosis site “S”. The anastomosis site can be created as part of any surgical procedure joining any two separated parts of an anatomical lumen, such as blood vessels, intestines, colon, and the like. Anastomosis may be performed using any surgical techniques using sutures, staplers, fasteners, adhesives, and any combinations thereof. The sensor assembly 12 is used for leak detection at the anastomosis site “S” or in the areas where another surgical intervention has greater risk of negative outcomes, such as in low anterior resection.

The sensor assembly 12 includes a sensor element 14 and a power and communications module 16, interconnected by a cable 13. The sensor element 14 may be a pH sensor configured to output a voltage or current signal indicative of pH at the site “S.” The sensor element 14 may be a potentiometric sensor configured to measure pH by using the electrical potential of pH-sensitive electrodes as the voltage signal. The cable 13 provides data and power transmission to and from the power module 16, respectively. Thus, the sensor element 14 provides sensor signals to the power and communications module 16, which provides power to the sensor element 14 and outputs sensor signals to a reader 20, which processes the signals as well as transmits sensor data to a computing device 30 for further processing. The computing device 30 may be a handheld device having a touchscreen and is configured to run an application for communicating with the sensor assembly 12, and in particular, with the power module/communication 16.

The sensor element 14 may be secured at the anastomosis site “S” using sutures, staplers, fasteners, meshes, films, adhesives, and combinations thereof which may be formed from a biodegradable material, such that after the material dissolves, the sensor element 14 detaches, and can then be extracted from the anastomosis site “S”. As used herein, the terms “biodegradable” and “bioabsorbable” are used with respect to a property of a material. “Biodegradable” is a material that is capable of being decomposed or broken down in vivo and subsequently excreted. “Bioabsorbable” is a material that is capable of being decomposed or broken down in vivo and subsequently resorbed. Both biodegradable and bioabsorbable materials are suitable for purposes of this application and thus for simplicity, unless otherwise directed, biodegradable materials and bioabsorbable materials are collectively referred to as “biodegradable” herein. Conversely, “non-biodegradable” is a biocompatible (i.e., not harmful to living tissue) material is not decomposed or broken down in vivo. In addition, the term “dissolution” as used in the description refers to the breakdown of both biodegradable and bioabsorbable materials.

The cable 13 allows for implantation of the power module 16 closer to the surface of a body, namely, subcutaneously and outside the peritoneum “P.” Proximity of the power and communications module 16 may enable stronger wireless communication with the computing device 30. The cable 13 may be passed through the peritoneum “P” through a small puncture, which may have been previously formed during anastomosis procedure. The power and communications module 16 may be an inductor coil which provides for data and power transmission. The inductor coil may be implanted using an awl device or pulled into subcutaneous space with a suture passer device.

The reader 20 is configured to periodically interrogate the power and communications module 16 to obtain sensor readings from the sensor assembly 12, which may be done from about every 10 seconds to about 1 hour. During interrogation, the reader 20 outputs a wireless power signal to energize the power and communications module 16, which in turn enable the sensor assembly 12 to take a pH reading and transmit the sensor through the power module 16 to the reader 20.

The reader 20 may be secured on the skin of the patient using adhesive tape or a belt. In embodiments, the reader 20 may be brought in proximity of the power module 16 to interrogate the power and communications module 16. Location of the power and communications module 16 may be marked with a temporary tattoo, a magnetic marker, detected by the reader 20 based on changes in amplitude during interrogation, and the like.

With reference to FIG. 2, the reader 20 includes a processor 21, which may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted by any logic processor (e.g., control circuit) adapted to execute algorithms, calculations, and/or set of instructions described herein.

The processor 21 may also include a memory device, which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory.

The reader 20 further includes a signal processor 23 and a battery 24, which may be any suitable rechargeable or non-rechargeable battery. The signal processor 23 receives sensor signals from the sensor assembly 12 and may include one or more analog-to-digital converters (ADC) and other signal processing circuit components to digitize the sensor signal for processing by the processor 21.

The reader 20 also includes a wireless interface 25, which may include an antenna and any other suitable transceiver circuitry configured to communicate with external devices (e.g., computing device 30) using wireless communication protocols. Wireless communication may be achieved via one or more wireless configurations, e.g., radio frequency, optical, Wi-Fi, ANT+, BLUETOOTH®, (an open wireless protocol for exchanging data over short distances, using short length radio waves, from fixed and mobile devices, creating personal area networks (PANs), ZIGBEE® (a specification for a suite of high level communication protocols using small, low-power digital radios based on the IEEE 802.15.4-2003 standard for wireless personal area networks (WPANs)), and the like. The wireless interface 25 may also include an inductor coil configured to couple to the counterpart inductor coil of the power module 16 to enable interrogation of the sensor assembly 12. This allows for the sensor assembly 12 to avoid the use of an internal power source, minimizing cost.

The reader 20 may also include an input device 26, which may include a display, i.e., a touchscreen, and/or one or more buttons, which allow for the user to control the computing device 30. In addition, the reader 20 includes an output device 27, which may be a display, a speaker, status LEDs, etc.

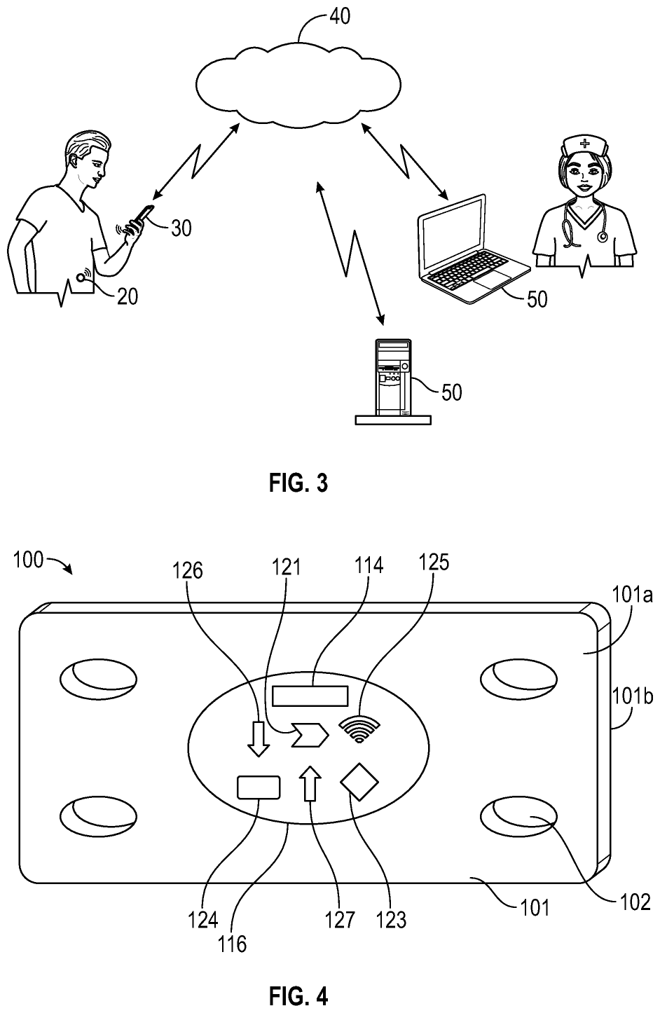

The reader 20 is configured to process sensor signals from the sensor assembly 12. Processing may include storing the sensor signals on a periodic basis, comparing the signals to thresholds, performing various calculations, including, rate of change calculations, average, minimum and maximum determinations, and the like. With reference to FIG. 3, the reader 20 may also transmit the sensor data to the computing device 30, which in turn may process the data locally and transmit the raw and/or processed sensor data to a cloud 40 (i.e., remote computing environment), which is accessible by other computing devices 50, which may be manufacturer's servers and health provider's computers. Data may be processed to detect anomalies in the pH at the computing device 30, the cloud 40, and/or the computing devices 50 and generate an alert on the computing device 30 to indicate to the patient to seek medical attention. The alert may also be output on a health provider's computing device 50 to notify that treatment may be required.

Anomaly detection may include comparing instantaneous, average, rates of change, and other pH-based values to predetermined thresholds and other parameters, which may be based on patient's historic pH values. A change in pH at the site above the patient's historic pH value or a range centered around the patient's historic pH value may be indicative of a possible anastomotic leak. In addition, pH values may be compared to predetermined thresholds, i.e., ranges, to determine the status of the healing process of the anastomosis site “S”, including the presence of an anastomotic leak.

The sensor data may be also analyzed to determine patterns in pH, which are indicative of anastomosis leaks, which may be identified using machine learning or other artificial intelligence algorithms. The identified patterns may be used by the computing devices 30 or 50 to detect anastomotic leaks. Machine learning may be performed on the cloud 40 and/or the computing devices 50 and the trained algorithms may be deployed on the computing device 30 and/or the computing devices 50 as firmware or software updates to improve early detection capabilities of the system 10.

The sensor assembly 12 may be implanted at the time of surgery for a leak-critical time period, which may be up to 14 days after surgery, after which the sensor assembly 12 is removed, which may be done in any suitable medical setting, e.g., outpatient facility. A small incision may be made to expose the power and communications module 16 and pull on the cable 13 to verify that the sensor element 14 is lose after biodegradable material securing the sensor element 14 to the site “S” has dissolved. After verification, the power module 16, along with the cable 13 and the sensor element 14 are removed and the incision closed (e.g. with a stitch, or glue).

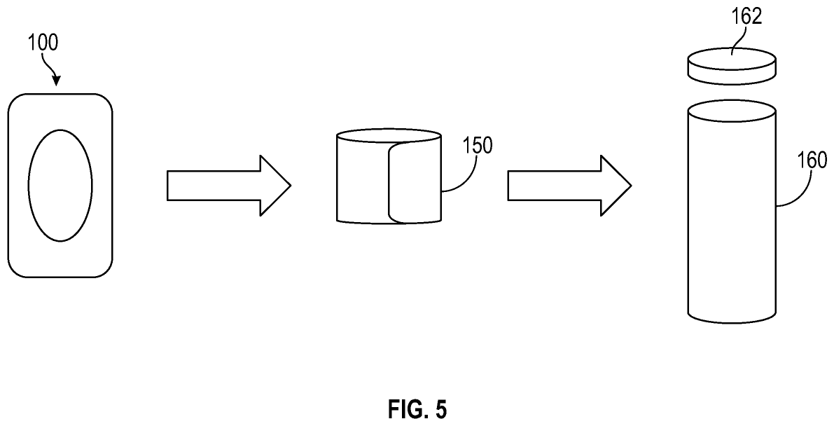

With reference to FIGS. 4 and 5 another embodiment of a sensor assembly 100 is shown, which includes a substrate 101, which may be formed from a biodegradable or nonbiodegradable biocompatible material. The substrate 101 may have any suitable shape, e.g., rectangular, circular, oval, polygonal, etc. and may be flexible, allowing for the substrate 101 to conform to the anastomosis site “S”. The substrate 101 may have a surface area from about 1 cm² to about 10 cm². In embodiments, the substrate 101 may be formed from a memory shape material allowing for the substrate 101 to transform from a first state to a second state in response to changes in temperature, namely, from room temperature of about 20° C. to body temperature of about 37° C. Suitable memory shape materials include metals, such as a nickel-titanium alloy (nitinol) or shape memory polymers, including (meth)acrylates, polyurethanes, polyvinylchlorides, and combinations thereof. At room temperature or lower, the substrate 101 is configured to transform into a roll 150 as shown in FIG. 5, whereas when exposed to a higher temperature, e.g., body temperature, the substrate 101 transforms into a substantially planar shape.

The substrate 101 includes a first surface 101a and a second opposing surface 101b. The substrate 101 may have a thickness from about 0.1 mm to about 5 mm. The substrate 101 may also include one or more openings 102, which are used as attachment points for securing the substrate 101 to the anastomosis site “S” using sutures or other fasteners described above. In embodiments, the substrate 101 may include an adhesive disposed on the first surface 101a. Adhesive may be used in combination with fasteners and/or sutures. The adhesive may have a peelable cover (not shown) that is removed prior to deployment of the sensor assembly 100.

The sensor assembly 100 includes a sensor element 114 disposed on the second surface 101b of the substrate 101, which is substantially similar to the sensor element 14 of the sensor assembly 12. The sensor assembly 110 also includes a circuit assembly 116, which includes the components of the reader 20. The circuit assembly 116 includes a processor 121, a signal processor 123, and a battery 124, which may be any suitable rechargeable or non-rechargeable battery. The signal processor 123 receives sensor signals from the sensor element 114 and may include one or more analog-to-digital converters (ADC) and other signal processing circuit components to digitize the sensor signal for processing by the processor 121.

The circuit assembly 116 also includes a wireless interface 125, which may include an antenna and any other suitable transceiver circuitry configured to communicate with external devices (e.g., computing device 30) using wireless communication protocols. The circuit assembly 116 may also include the input device 126, which may be a port allowing for communication with the processor 121 and the output device 127, which may be status LEDs, etc. The circuit assembly 116 is configured to process sensor signals from the sensor element 114. Processing may include storing the sensor signals on a periodic basis, comparing the signals to thresholds, annotating sensor signals of interest, and the like. The sensor assembly 100 may be continuously or periodically interrogated by the reader 20 and/or the computing device 30 to process, analyze, and/or transmit sensor data as described above with respect to FIGS. 1 and 3.

The sensor assembly 100 may be implanted through a small incision, which may be formed specifically for implanting the sensor assembly 100 or to reuse an incision used in the anastomosis procedure. The sensor assembly 100 may be shaped into the roll 150 either using mechanical force or by adjusting the temperature of the substrate 101 if the substrate 101 is formed from a memory shape material. Once the roll 150 is formed, the roll 150 is inserted to the anastomosis site “S”. The roll 150 may be inserted through a trocar (not shown). Once at the anastomosis site “S”, the substrate 101 is unfurled into the substantially planar shape and the substrate 101 is secured to the anastomosis site “S.” This may be done by exposing the adhesive disposed on the first surface 101a and attaching the first surface 101a to the tissue as well as using sutures and/or other fasteners to secure the substrate 101 through the openings 102.

Since the adhesive and the sutures are formed from biodegradable material, after a predetermined period of time has passed, which may be from about 5 days to about 14 days, the sensor assembly 100 may be removed through the incision. A retrievable capsule 160 may be inserted to the anastomosis site. The retrievable capsule 160 may be rigid or flexible and may have a substantially cylindrical shape to receive the roll 150 therein and may have a cover 162. The sensor assembly 100 is removed from the anastomosis site “S” by peeling the substrate 101 from the tissue. The sensor assembly 100 may be rolled up into the roll 150 by using mechanical force and/or lowering the temperature. Once the sensor assembly 100 is rolled up, the roll 150 may be placed in the retrievable capsule 160, which may then be removed. Alternatively, the roll 150 may be passed directly through a trocar, obviating the need for the capsule 160.

After retrieval, the data stored on the sensor assembly 100 may be further analyzed to determine patterns in pH, which are indicative of anastomosis leaks, which may be identified using machine learning or other artificial intelligence algorithms. The identified patterns may be used by the computing devices 30 or 50 to detect patterns corresponding anastomotic leaks. Machine learning may be performed on the cloud 40 and/or the computing devices 50 and the trained algorithms may be deployed on the computing device 30 and/or the computing devices 50 as firmware or software updates to improve early detection capabilities of the system 10.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A system for detecting an anastomotic leak, the system comprising:
a sensor assembly implanted at an anastomosis site;
a reader configured to receive sensor signals from the sensor assembly; and
a computing device configured to communicate with the reader, the computing device configured to analyze the sensor signals to determine a status of the anastomosis site.

2. The system according to claim 1, wherein the sensor assembly includes:
a sensor element configured to measure pH of the anastomosis site.

3. The system according to claim 2, wherein the sensor assembly further includes:
a power and communications module coupled to the sensor element via a cable.

4. The system according to claim 3, wherein the power and communications module is implanted subcutaneously and outside the peritoneum.

5. The system according to claim 4, wherein the computing device is configured to compare the sensor signals to a pH range.

6. The system according to claim 4, wherein the computing device is configured to detect a pattern in the sensor signals indicative of an anastomotic leak.

7. The system according to claim 2, wherein the sensor assembly includes:
a substrate having a first surface and a second surface and a substantially planar shape.

8. The system according to claim 7, wherein the substrate includes at least one opening configured to be used as an attachment point.

9. The system according to claim 1, wherein the computing device is configured to communicate with a remote server.

10. The system according to claim 9, wherein the remote server is configured to analyze the sensor signals to determine the status of the anastomosis site.

11. The system according to claim 1, wherein the reader is further configured to wirelessly transmit power to the sensor assembly.

12. A method for detecting an anastomotic leak, the method comprising:
implanting a sensor assembly at an anastomosis site;
receiving wireless sensor signals from the sensor assembly at a reader; and
analyzing the sensor signals to determine a status of the anastomosis site.

13. The method according to claim 12, further includes:
measuring pH at the anastomosis site.

14. The method according to claim 13, wherein implanting the sensor assembly includes implanting a sensor element at the anastomosis site.

15. The method according to claim 14, wherein implanting the sensor assembly includes implanting a power and communications module subcutaneously and outside the peritoneum, wherein the power module is coupled to sensor element via a cable.

16. The method according to claim 12, further comprising:
comparing the sensor signals to a pH range.

17. The method according to claim 12, further comprising:
detecting a pattern in the sensor signals indicative of an anastomotic leak; and
notifying a health care professional of the anastomotic leak.

18. A sensor assembly for detecting an anastomosis leak, the sensor assembly comprising:
a substrate having a first surface and a second surface and a substantially planar shape; and
a sensor element configured to measure pH disposed on the substrate.

19. The sensor assembly according to claim 18, wherein the substrate includes at least one opening configured to be use as an attachment point.

20. The sensor assembly according to claim 18, further comprising an adhesive disposed on at least one of the first surface or the second surface.
